(19) United States
(12) Patent Application Publication
(10) Pub. No.: US 2006/0182820 A1
(43) Pub. Date: Aug. 17, 2006

(54) Homogeneous Dispersions Containing Citrus Pulp and Applications Thereof

(75) Inventors: Michael D. Kluetz, Watertown, MN (US); Melanie J. Goulson, Dayton, MN (US); Vincent M. Cavallini, Shakopee, MN (US); Wade N. Schmelzer, St. Louis Park, MN (US)

Correspondence Address: Dorothy P. Whelan;FISH & RICHARDSON P.C., P.O.BOX 1022, MINNEAPOLIS, MN, 55440-1022, US

(73) Assignee: Cargill, Incorporated, Wayzata, MN (US)

(21) Appl. No.: 11/380,581

(22) Filed: Apr. 27, 2006

Related U.S. Application Data
(63) Continuation-in-part of application No. 10/775,933, filed on Feb. 10, 2004.

(51) Int. Cl. A61K 36/752

(52) U.S. Cl. 424/736

(57) ABSTRACT

A composition in the form of a homogeneous dispersion that includes citrus pulp and a plant sterol, omega-3-oil, and/or isoprenoid. The dispersion may be incorporated in foods and beverages.

### STATEMENT OF RELATED CASES

This application is a continuation-in-part of Kluetz et al., U.S. Ser. No. 10/775,933 filed Feb. 10, 2004 and entitled “Particulate Plant Sterol Compositions,” the contents of which are hereby incorporated by reference in their entirety.

### TECHNICAL FIELD

This invention relates to preparing homogeneous dispersions using citrus pulp.

### BACKGROUND

Plant sterols are known to reduce total and LDL cholesterol, and to reduce the risk of cardiovascular disease. Accordingly, attempts have been made to incorporate plant sterols into food products containing fats and oils. Because the sterols, like the fats and oils, are hydrophobic, the incorporation has been accomplished with little adverse sensory impact. However, attempts to incorporate plant sterols into hydrophilic products such as beverages have been less successful because the sterols visibly separate from the remainder of the composition. In addition to the poor aesthetic appearance, the composition may leave residue in containers and glasses, and may have a chalky or powdery texture or mouthfeel, as well as other undesirable taste effects.

### SUMMARY

A composition in the form of a homogeneous dispersion that includes citrus pulp and a plant sterol, omega-3-oil, and/or isoprenoid is described. Preferably, the dispersion is essentially free of citrus juice concentrate. The dispersion may be incorporated in foods and beverages with little adverse sensory impact, thereby providing a convenient means for delivering plant sterols, omega-3-oils, and/or isoprenoids to a consumer.

The details of one or more embodiments of the invention are set forth in the accompanying description below. Other features, objects, and advantages of the invention will be apparent from the detailed description and from the claims.

### DETAILED DESCRIPTION

A plant sterol, an omega-3-oil, an isoprenoid (e.g., coenzyme Q₁₀, carotenoids, limonoids, lutein, and the like), or combination thereof, is combined with citrus pulp to form a homogeneous dispersion. The dispersion preferably is essentially free of citrus juice concentrate. The dispersion can be added to beverages or foods. The beverages may be in the form of juices or juice concentrates.

As used herein, the term “homogeneous dispersion” refers to a composition having a uniform composition throughout in which the components are too intimately combined to be distinguishable by visual observation.

As used herein, the term “plant sterols” refers to phytosterols, phytosterol esters, phytostanols, and phytostanol esters.

As used herein, the term “citrus pulp” refers to the insoluble material found in the edible section of a citrus fruit. It includes juice sacs (whole and ruptured) and segment walls.

Plant sterols for use herein can include any of various positional isomer and stereoisomeric forms, such as α-, β-, or γ-isomers. Typical phytosterols include α-sitosterol, β-sitosterol, γ-sitosterol, campesterol, stigmasterol, brassicasterol, spinosterol, taraxasterol, desmosterol, chalinosterol, poriferasterol, clionasterol, ergosterol, Δ-5-avenosterol, Δ-5-campesterol, clerosterol, Δ-5-stigmasterol, Δ-7,25-stigmadienol, Δ-7-avenosterol, Δ-7-β-sitosterol, and Δ-7-brassicasterol.

Suitable examples of phytosterol esters include β-sitosterol laurate ester, α-sitosterol laurate ester, γ-sitosterol laurate ester, campesterol myristearate ester, stigmasterol oleate ester, campesterol stearate ester, β-sitosterol oleate ester, β-sitosterol palmitate ester, β-sitosterol linoleate ester, α-sitosterol oleate ester, γ-sitosterol oleate ester, β-sitosterol myristearate ester, β-sitosterol ricinoleate ester, campesterol laurate ester, campesterol ricinoleate ester, campesterol oleate ester, campesterol linoleate ester, stigmasterol linoleate ester, stigmasterol laurate ester, stigmasterol caproate ester, α-sitosterol stearate ester, γ-sitosterol stearate ester, α-sitosterol myristearate ester, γ-sitosterol palmitate ester, campesterol ricinoleate ester, stigmasterol ricinoleate ester, campesterol ricinoleate ester, and stigmasterol stearate ester.

Useful phytostanols include α-, β-, and γ-sitostanol, campestanol, stigmastanol, spinostanol, taraxastanol, brassicastanol, desmostanol, chalinostanol, poriferastanol, clionastanol, and ergostanol.

Examples of phytostanol esters include β-sitostanol laurate ester, campestanol myristearate ester, stigmastanol oleate ester, campestanol stearate ester, β-sitostanol oleate ester, β-sitostanol palmitate ester, β-sitostanol linoleate ester, β-sitostanol myristearate ester, β-sitostanol ricinoleate ester, campestanol laurate ester, campestanol ricinoleate ester, campestanol oleate ester, campestanol linoleate ester, stigmastanol linoleate ester, stigmastanol laurate ester, stigmastanol caproate ester, stigmastanol stearate ester, α-sitostanol laurate ester, γ-sitostanol laurate ester, α-sitostanol oleate ester, γ-sitostanol oleate ester, α-sitostanol stearate ester, γ-sitostanol stearate ester, α-sitostanol myristearate ester, γ-sitostanol palmitate ester, campestanol ricinoleate ester, stigmastanol ricinoleate ester, campestanol ricinoleate ester, β-sitostanol, α-sitostanol, γ-sitostanol, campestanol, and stigmastanol.

Plant sterols can be derived from a variety of plant sources, including rice bran oil, corn fiber oil, corn germ oil, wheat germ oil, sunflower oil, safflower oil, oat oil, olive oil, cotton seed oil, soybean oil, peanut oil, canola oil, tea, sesame seed oil, grapeseed oil, rapeseed oil, linseed oil, tall oil and other oils obtained from wood pulp, and various other brassica crops. Although plant sterols are typically derived from plants, a plant sterol can also be synthetically prepared, e.g., it need not be derived from a plant source. Additionally, plant sterols can be prepared as mixtures of individual purified or synthesized plant sterol compounds or can be co-products resulting from purifications of other products (e.g., from plant sources). For example, a plant sterol can be obtained as a co-product of the manufacture of vitamin E and/or tocopherols from vegetable oil deodorizer distillate.

Plant sterols can be in any form, e.g., pastilles, prills, granules, or powders. Plant sterols can be obtained commercially from a number of sources, including Cargill, Incorporated (Minneapolis, Minn.), Cognis Nutrition and Health (La Grange, Ill.), Forbes Meditech (Vancouver, B.C. Canada), and ADM (Decatur, Ill.). Also suitable are plant sterols having a multi-peak volume-weighted or mass-weighted particle size distribution, as described in certain embodiments of the aforementioned Kluetz et al. application.

The citrus pulp may be produced from any citrus fruit. Non-limiting examples include orange, lime, lemon, grapefruit, tangerine, citrange, tangelo, pomelo, mandarin, citron, and kumquat pulp, and combinations thereof. Suitable forms of citrus pulp for use in preparing the homogenized dispersions include aseptic pulp, frozen pulp, washed pulp, and dried pulp.

Aseptic pulp may be prepared by passing a juice stream through primary finishers. The back-pressure settings on the pulp finishers will generally be quite low so that the pulp obtained still has high juice content. The resulting aseptic pulp is roughly 50% juice and 50% pulp solids, and is pasteurized before packaging.

Washed pulp is generally produced in the same manner as aseptic pulp. The pulp typically undergoes additional finishing steps after pasteurization to remove additional juice and liquid, after which the pulp is packaged and frozen. Typically, in a 3- or 4-stage countercurrent finishing train, about 60% of the total “solids from pulp” are removed as soluble orange solids, the remaining 40% comprising true “pulp solids (insolubles).”

The dispersion can be prepared by homogenizing a mixture of citrus fruit pulp and a plant sterol, omega-3-oil, and/or isoprenoid to produce a smooth, creamy composition. Water may be included in the homogenization process to help fluidize the pulp. The plant sterol, omega-3-oil, and/or isoprenoid can be included at about 0.1% to about 11% (e.g., 1% to 10%) by total weight of a water/pulp (solids)/sterol, omega-3-oil, and/or isoprenoid mixture prior to homogenization, or any value in between. In certain cases, the plant sterol, omega-3-oil, and/or isoprenoid can be included at about 3-7% or 2-3% by weight.

The dispersion can be formed by mixing the pulp, plant sterol, omega-3-oil, and/or isoprenoid, and, optionally, water prior to homogenization. For example, a pulp, water, and a plant sterol, omega-3-oil, and/or isoprenoid can be pre-mixed with high shear mixer. Premixing can occur until the plant sterol, omega-3-oil, and/or isoprenoid is well-dispersed in the pulp/water mixture. It may also be advantageous to pre-shred the pulp before dilution and incorporation of the plant sterol, omega-3-oil, and/or isoprenoid to facilitate its incorporation. The extent of pre-shredding can be estimated from the percentage of treated pulp that passes through an ASTM #30 or #40 screen upon extensive washing with a stream or spray of water (sometimes referred to as a “quick fiber” test).

Examples of high-shear equipment available to accomplish pre-shredding and pre-mixing include bench-top equipment such as a PowerGen® or IKA Ultra-Turrax®, and process equipment such as a Waukesha TriBlender®), Quadro/Ytron® Y, Z, or ZC, Urschel Comitrol®, and IKA Models MDH® or DRS® and the like.

The resulting mixture can then be homogenized. The mixture can be homogenized in more than one stage. For example, the mixture can be homogenized in two stages. In certain cases, the mixture is homogenized at about 3000-5000 psi in a first stage, and then homogenized at about 300-800 psi in a second stage. In some cases, the mixture is homogenized at 4500 psi in a first stage and 500 psi in a second stage. Multiple passes can also be used.

After homogenization, the homogeneous dispersion can be incorporated in a food or beverage by simple mixing or by the procedure described in the Examples. For example, it can be incorporated in beverages such as a fruit juice (such as orange, grape, cranberry, apple, kiwi, mango, peach, pineapple, plum, cherry, banana, guava, papaya, grapefruit, natsudaidai, tangerine, clementine, mandarin orange, currant, watermelon, honeydew melon, cantaloupe, lemon, lime, pear, blueberry, blackberry, raspberry, or strawberry juice), or a vegetable juice (such as tomato, carrot, celery, cucumber, spinach, lettuce, watercress, sprouts, beet, herbs, cabbage, or wheat grass juice, or mixtures of juices), a juice concentrate, coffee, tea, a smoothie, a shake, soy milk, rice milk, a frappe, a milk fluid (e.g., full fat milk, 1% milk, 2% milk, skim milk, heavy cream, half and half, whipping cream, or light cream), a meal replacement beverage, a diet beverage, or a nutritional supplement beverage. Examples of suitable beverages include fruit nectar, fruit juice, vegetable juice, cocktail, or smoothie.

The dispersion can also be incorporated in a food composition, such as ice cream, yogurt, gels, or a nutritional supplement. In certain food compositions, the dispersion can be incorporated into an aqueous composition, which is then incorporated into a solid food composition (such as baked goods). The composition may also be a variety of fruit-based compositions including fruit preparations (including those used in fruit yogurt), jams, jellies, fruit leathers, fruit sauces, fruit chews, confections, and the like.

The beverage and food compositions can be formulated such that they contain about 0.3 g to about 1.8 g (or any value therebetween, e.g., about 0.4, 0.5, 0.6, 0.7, 0.8, 0.9, 1.0, 1.1, 1.2, 1.3, 1.4, 1.5, 1.6, or 1.7) of plant sterols per serving; 5-50 mg of isoprenoids per serving; or 25-250 mg of omega-3-oil per serving. A serving is typically 6-12 oz.

“Total sterol basis” is the additive sterol level from sterols and sterol esters. During the production of sterol esters, sterols are esterified with various fatty acids from many different sources and or blends. In order to determine the sterol contribution from a sterol ester product, the average molecular weights of the sterols and fatty acids are used to develop a conversion factor (to a free sterol basis) relative to the average molecular weight of the sterols divided by the average molecular weight of the sterols plus the average molecular weight of the fatty acids. The contribution from sterols and sterol esters are then added to obtain the total sterol basis. “Total stanol basis” is calculated in a similar manner for stanols and stanol esters. For example, 3% sterol esters with canola fatty acids equates to 1.875% by weight on a total sterol basis, as the conversion factor for those sterol esters is 0.625. If the 3% level was obtained by sterols used in combination with sterol esters, in a 50:50 ratio, then the total sterol basis would be 2.4375% by weight.

### EXAMPLES

### Example 1

The following process was used to produce orange juice containing 1 g particulate plant sterols per 240 mL:

Pulp Preparation

| Ingredient | % (w/w) As Is | % solids |
|---|---|---|
| Water | 24.30 | 0.0 |
| Orange pulp | 72.89 | 3.6 |
| Plant sterols | 2.81 | 2.8 |
| Total | 100.00 | 6.4 |

The orange pulp was in the form of frozen unwashed pulp cells (Valencia orange pulp cells, available from Cargill, Incorporated). The unwashed pulp cells had a moisture content of about 85% w/w. The plant sterols were CoroWise™ FP100, available from Cargill, Incorporated.

The pulp, water, and sterol mixture was premixed with high shear (10,000 rpm) for 5 minutes using a bench top high shear mixer (PowerGen® 1800D, Fisher Scientific). The premixed pulp, water, and sterol mixture was then homogenized (bench top homogenizer Model 15, APV Gaulin, Inc.) in two stages at 4500/500 psi.

Next, a single strength orange juice was prepared as follows:
1. Blend orange juice concentrate and water in a high shear mixer (PowerGen 1800D, Fisher Scientific) at 10,000 rpm.
2. Add sterol pulp dispersion under high shear conditions.
3. Thermally process the mixture on a MicroThermics unit by heating the beverage mixture to 190° F. Subject the heated mixture to a single pass through an in-line two-stage Niro homogenizer (Model 15, Niro, Inc.), with settings of 2500 psi/500 psi on the first and second stages, respectively.
4. Hot fill into bottles, seal, then invert for 2 minutes.
5. Cool the orange juice in an ice bath.

The juice ingredients are listed in the following table:

| Ingredient | % (w/w) As Is |
|---|---|
| Water | 65.41 |
| Sterol-containing Pulp Preparation | 16.46 |
| Frozen Concentrated Orange Juice | 18.13 |
| Total | 100.00 |

This formula delivered about 1 g of plant sterols in an 8 fluid ounce (240 ml) serving of juice.

### Example 2

A series of homogeneous dispersions were created using frozen, unwashed pulp cells (Valencia Orange frozen pulp cells, available from Cargill, Incorporated), fine particle plant sterols (CoroWise™ FP100 available from Cargill, Incorporated), and water, as shown below. The dispersions were prepared according to the procedure described in Example 1. All of the resulting dispersions had a pH around 4.0/4.1, and a very creamy white texture and appearance.

| Formulation | | | |
|---|---|---|---|
| % (w/w) As Is | A | B | C |
| Water | 76.0% | 71.3% | 77.6% |
| Orange pulp cells | 19.0% | 23.7% | 19.4% |
| Plant sterols | 5.0% | 5.0% | 3.0% |
| Total Solids from Pulp* | 2.8% | 3.6% | 2.9% |
| Ratio - Sterols to Total Solids | 1.8 | 1.4 | 1.0 |
| from Pulp | | | |
| Estimated Ratio of Sterols to Pulp | 4.4 | 3.5 | 2.6 |
| Insolubles^{†} | | | |
| Ratio - Water to Total Solids | 32.3 | 25.7 | 32.3 |
| from Pulp | | | |

*Based on 85.0% moisture content for unwashed, orange pulp cells.
^{†}Based on estimate that ˜60% of total solids from pulp are water-extractable, soluble orange solids, and only ˜40% is actual pulp insolubles.

### Example 3

6. Single strength orange juices were prepared using the dispersions described in Example 2. The orange juices were prepared according to the procedure described in Example 1, with a target of 0.4 grams of plant sterols in an 8 fluid ounce (240 mL) serving of juice.

| Formulation | A | B | C |
|---|---|---|---|
| Water | 78.25% | 78.25% | 75.82% |
| Orange Juice Conc (65° Brix) | 18.00% | 18.00% | 18.00% |
| Sterol Pulp Dispersion ‘A’ (Ex 2) | 3.75% | | |
| Sterol Pulp Dispersion ‘B’ (Ex 2) | | 3.75% | |
| Sterol Pulp Dispersion ‘C’ (Ex 2) | | | 6.18% |

Similarly, orange juices using higher levels of the sterol pulp dispersions from Example 2 can be produced to achieve a target level of 1.0 grams of plant sterols in an 8 fluid ounce (240 mL) serving of juice (see below).

| Formulation | A | B | C |
|---|---|---|---|
| Water | 72.60% | 72.60% | 66.57% |
| Orange Juice Conc (65° Brix) | 18.00% | 18.00% | 18.00% |
| Sterol Pulp Dispersion ‘A’ (Ex 2) | 9.40% | | |
| Sterol Pulp Dispersion ‘B’ (Ex 2) | | 9.40% | |
| Sterol Pulp Dispersion ‘C’ (Ex 2) | | | 15.43% |

### Example 4

A juice drink containing 70% juice utilized a sterol pulp dispersion with a composition of 76% water, 19% unwashed orange pulp cells and 5% fine particle plant sterols (CoroWise™ FP100 available from Cargill, Incorporated) was prepared. The formula delivers about 0.4 grams of plant sterols in an 8 fluid ounce (240 mL) serving of beverage. The juice concentrates, fruit puree and high fructose corn syrup were mixed into the water, followed by addition of the sterol pulp dispersion. After addition of the flavor, the juice drink was thermally processed as described in Example 1.

| Ingredient | % (w/w) As Is |
|---|---|
| Water | 71.05 |
| High Fructose Corn Syrup (55 HFCS) | 10.00 |
| Orange Juice Concentrate (65° Brix) | 6.90 |
| Pineapple Juice Concentrate (60° Brix) | 4.00 |
| Banana Puree | 3.45 |
| Lemon Juice Concentrate (44° Brix) | 0.75 |
| Sterol Pulp Dispersion | 3.75 |
| Pineapple Flavor | 0.05 |
| Banana Flavor | 0.05 |
| Total | 100.00 |

### Example 5

A single strength cloudy apple juice also utilizing a sterol pulp dispersion with a composition of 76% water, 19% unwashed orange pulp cells and 5% fine particle plant sterols (CoroWise™ FP100 available from Cargill, Incorporated) was prepared. The formula delivers about 0.4 grams of plant sterols in an 8 fluid ounce (240 mL) serving of beverage. The apple juice was prepared and thermally processed as describe in Example 1.

| Ingredient | % (w/w) As Is |
|---|---|
| Water | 67.50 |
| Cloudy Apple Juice Concentrate (40° Brix) | 28.75 |
| Sterol Pulp Dispersion | 3.75 |
| Total | 100.00 |

### Example 6

The following dispersions were prepared using washed orange pulp cells (obtained from Cargill, Incorporated), fine particle plant sterols (CoroWise™ FP100 available from Cargill, Incorporated), and water. Dispersions A and B were prepared using the procedure described in Example 1.

| | A | | B | |
|---|---|---|---|---|
| | % (w/w) | % | % (w/w) | % |
| Ingredient | As Is | Solids | As Is | Solids |
| Water | 76.0% | 0 | 75.0% | 0 |
| Orange pulp cells | 19.0% | 1.65% | 19.0% | 1.1% |
| Plant sterols | 5.0% | 5.00% | 6.0% | 6.0% |
| Total | 100.0% | 6.65% | 100.0% | 7.1% |
| Ratio - Sterols to Total | — | 3.0 | — | 5.4 |
| Pulp Insolubles | | | | |
| Ratio - Water to Total | — | 56.6 | — | 84.4 |
| Pulp Insolubles | | | | |

The following dispersion was prepared using laboratory-prepared grapefruit pulp cells and CoroWise™ FP-100 Powdered Sterols using the procedure of Example 1. Four large grapefruit weighing about 5 lb were peeled and passed through a kitchen grinder;

the resulting mass was washed though a #12 screen to remove membrane and seed fragments, and the pulp was then collected and washed on a #18 screen, followed by dewatering with a 100-mesh fiber filter under vacuum. Approximately 225 gm of pulp was obtained at a moisture content of 95%.

| Ingredient | % (w/w) As Is | % Solids |
|---|---|---|
| Water | 66.3% | 0 |
| Grapefruit pulp cells | 27.7% | 1.4% |
| Plant sterols | 6.0% | 6.0% |
| Total | 100.0% | 7.4% |
| Ratio - Sterols to Total Pulp Insolubles | — | 4.3 |
| Ratio - Water to Total Pulp Insolubles | — | 66 |

### Example 7

A dispersion was prepared by homogenizing water, cod liver oil, which contains about 25% EPA/DHA omega-3 fatty acids, and orange juice pulp according to the procedure described in Example 1 except that sterols were replaced by the cod liver oil. The dispersion was used to prepare an orange juice formulation according to the procedure described in Example 1. The juice was designed to provide 85.0 mg of EPA/DHA per 240 g serving of juice, and had the following formulation:

| Ingredients | % (w/w) As Is | Grams |
|---|---|---|
| Water | 72.6 | 3631.5 |
| EPA/DHA pulp dispersion | 9.4 | 468.5 |
| Orange juice concentrate | 18.0 | 900 |
| Total | 100.00 | 5000 |

After 24 hours, the juice appeared stable and the omega-3 containing oil had not separated out. The same results were observed after one month. Other examples of omega-3 rich oils that could be used include flaxseed, algal, menhaden and other fish oils.

### Example 8

In one case unwashed pulp was shredded by passage through an Urschel Comitrol® Processor Model 1700 prior to dilution and incorporation of the sterols; the other dispersion was prepared by pre-shredding approximately 950 lb of washed pulp by recirculation through a Waukesha TriBlender® high-shear pump, diluting with 350 lb water, then incorporating 60 lb sterols using the same unit, followed by homogenization at 4500/500 psi on a Gaulin homogenizer. Washed pulp cells have a higher moisture content as a result of the washing process, and varies considerably depending on the back-pressure setting of the finishers, as can be seen from the high moisture of the washed pulp used in this Example.

| | Unwashed Pulp with | | Washed Pulp with | |
|---|---|---|---|---|
| | Comitrol ® | | TriBlender ® | |
| | Processing | | Processing | |
| | % (w/w) | | % (w/w) | |
| Ingredient | As Is | % Solids | As Is | % Solids |
| Water | 60.1% | 0 | 24.9% | 0 |
| Orange pulp cells* | 29.9% | 4.0% | 70.7% | 1.6% |
| Plant sterols | 11.0% | 11.0% | 4.4% | 4.4% |
| Ratio - Sterols to Total | — | 2.75 | — | 2.75 |
| Solids from Pulp | | | | |
| Estimated Ratio of | — | 6.9 | — | n.a. |
| Sterols to Pulp Insolubles | | | | |
| Ratio - Water to Total | — | 21.2 | — | 58.8 |
| Solids from Pulp | | | | |

*Comitrol ® pulp at 86.5% moisture; plant-washed pulp used with TriBlender ® at 97.7% moisture

A number of embodiments of the invention have been described. Nevertheless, it will be understood that various modifications may be made without departing from the spirit and scope of the invention. Accordingly, other embodiments are within the scope of the following claims.

What is claimed is:

1. A composition comprising a plant sterol and citrus pulp, wherein the composition is in the form of a homogeneous dispersion.

2. The composition of claim 1, wherein the composition is essentially free of citrus juice concentrate.

3. The composition of claim 1 wherein the citrus pulp is selected from the group consisting of orange, lime, lemon, grapefruit, tangerine, citrange, tangelo, pomelo, mandarin, citron, and kumquat pulp, and combinations thereof.

4. The composition of claim 3 wherein the citrus pulp comprises orange pulp.

5. The composition of claim 1 comprising between about 1% and about 10% of the plant sterol based upon the total weight of the composition.

6. The composition of claim 1 wherein the plant sterol demonstrates a multi-peak, volume- or mass-weighted particle size distribution.

7. The composition of claim 1 wherein the weight ratio of plant sterol to citrus pulp insolubles is from about 2:1 to about 7:1.

8. The composition of claim 1 wherein the plant sterol is derived from a plant source selected from the group consisting of rice bran oil, corn fiber oil, corn germ oil, wheat germ oil, sunflower oil, safflower oil, oat oil, olive oil, cotton seed oil, grapeseed oil, rapeseed oil, linseed oil, tall oil, and combinations thereof.

9. The composition of claim 1 wherein the plant sterol is derived from tall oil.

10. A method for preparing a dispersion of a plant sterol composition, comprising homogenizing a plant sterol composition with citrus pulp.

11. The method of claim 10 wherein said homogenizing step occurs in the absence of citrus juice concentrate.

12. The method of claim 10 wherein said homogenizing step further includes water.

13. The method of claim 12 wherein prior to said homogenizing, said pulp, said water, and said sterol composition are mixed with high shear.

15. The method of claim 12 wherein said plant sterol composition is about 1% to about 10% by weight of said total amount of said pulp, said water, and said plant sterol composition.

16. A method for preparing a food or beverage composition comprising mixing a homogeneous dispersion comprising a plant sterol and citrus pulp with a food or beverage.

17. The method of claim 16 wherein the homogeneous dispersion is essentially free of citrus juice concentrate.

18. The method of claim 16 comprising mixing the homogeneous dispersion with a beverage.

19. The method of claim 18 wherein the beverage comprises a juice or juice concentrate.

20. The method of claim 19 wherein the beverage comprises a fruit juice or juice concentrate.

21. The method of claim 20 wherein the fruit juice or juice concentrate comprises orange juice or juice concentrate.

22. The method of claim 20 wherein the fruit juice or juice concentrate is derived from a fruit that is different from the fruit from which the citrus pulp is derived.

23. The method of claim 16 comprising mixing the homogeneous dispersion with a food.

24. The method of claim 16 wherein the amount of plant sterol in the food or beverage composition is selected to deliver about 0.3 g to about 1.8 g of plant sterol per 6-12 oz. serving of the food or beverage composition.

25. A food or beverage composition prepared according to the method of claim 16.

26. A food or beverage composition prepared according to the method of claim 19.

27. A composition comprising (a) an agent selected from the group consisting of omega-3-oils, isoprenoids, and combinations thereof and (b) citrus pulp, wherein the composition is in the form of a homogeneous dispersion.

28. The composition of claim 27, wherein the composition is essentially free of citrus juice concentrate.

29. A method for preparing a dispersion of an agent selected from the group consisting of omega-3-oils, isoprenoids, and combinations thereof, comprising homogenizing the agent with citrus pulp.

30. The method of claim 29 wherein said homogenizing step occurs in the absence of citrus juice concentrate.

31. A method for preparing a food or beverage composition comprising mixing a homogeneous dispersion comprising (a) an agent selected from the group consisting of omega-3-oils, isoprenoids, and combinations thereof and (b) citrus pulp, with a food or beverage.

32. The method of claim 31 wherein the homogeneous dispersion is essentially free of citrus juice concentrate.
